(19) United States
(12) Patent Application Publication
(10) Pub. No.: US 2016/0228719 A1
(43) Pub. Date: Aug. 11, 2016

(54) METHOD FOR TREATING NEURODEGENERATIVE DISEASE USING LOW-INTENSITY RADIO-FREQUENCY ELECTROMAGNETIC WAVES

(71) Applicants: Salvatore RINALDI, Scandicci (IT); Vania FONTANI, Scandicci (IT)

(72) Inventors: Salvatore RINALDI, Scandicci (IT); Vania FONTANI, Scandicci (IT)

(21) Appl. No.: 15/016,617

(22) Filed: Feb. 5, 2016

Related U.S. Application Data
(60) Provisional application No. 62/112,728, filed on Feb. 6, 2015.

(51) Int. Cl. A61N 1/40; A61N 5/02

(52) U.S. Cl. CPC A61N 1/40; A61N 5/022

(57) ABSTRACT

The invention relates to a method for treating a neurodegenerative disease. The treatment involves administering a series of pulses and pauses from a radio frequency generator to a patient with a neurodegenerative disease. Parkinson's Disease, Alzheimer's Disease, and Post Stroke Disorder are among the conditions which can be treated,

### FIELD OF THE INVENTION

The present invention relates to the technical field of methods and devices for curative and symptomatic treatments in particular for neurodegenerative disease.

### PRIOR ART

In the technical field of curative and symptomatic treatment using devices provided with electrodes for electro-stimulation and electrotherapy, said electrodes are usually suitable for irradiating a part of the patient's body so as to localise the intervention where necessary.

Examples of devices of this type are disclosed in documents U.S. Pat. No. 4,121,592, U.S. Pat. No. 4,197,851, U.S. Pat. No. 5,507,743 and EP1301241.

Document U.S. Pat. No. 4,121,592 discloses a device for heating fabrics, which can be used for treating tumours in humans and animals. In this device, an electrical field is produced between two electrodes and applied to the tissue region including and surrounding the tumour. The polarity and intensity of the applied radiofrequency electrical field are varied depending on the type of illness to be treated.

Document U.S. Pat. No. 4,197,851 discloses a device for emitting high-frequency electromagnetic waves, comprising a power supply and an antenna comprising at least one conductor and a flexible insulating support such that it can be shaped so as to be applied directly to parts of the human body. The antenna may be linked to current-emitting electrodes, and said current may be emitted alternately or simultaneously with the electromagnetic field emission brought about by the antenna.

Document U.S. Pat. No. 5,507,743 discloses a further device for treating tumorous tissues, comprising a pair of electrodes, one active and one passive. The active electrode may be in the form of a probe which can be introduced into the tissue to be treated, whilst the passive electrode may be in an extended form so as to be applied around the region involved in the treatment. The passive electrode is coupled to a microwave generator, whilst the active electrode is connected to a power supply so as to act as an ablation electrode.

Document EP1301241 discloses a device for treating patients which is based on a radiofrequency generator provided with one or more electrodes connected to the earth connection of the generator. Said electrode hereinafter called ACP (asymmetric conveyer probe) acts so as to direct the electromagnetic current induced in the generator into the patient's body in a suitable manner.

Said electrodes, depending on the requirements of the treatment to be applied, in the form of leads, probes, plates or extended conductive sheets.

In this last case, one of the treatments which can be applied to the patient provides the use of extended conductive electrodes which are suitable for application to large portions of the patient's body. As stated, these electrodes are suitable for directing, into the patient's body, the electromagnetic currents induced by the electromagnetic field generated by the device in the space surrounding the actual patient.

However, it is clear that when the electrodes have an extended surface area, so as to be suitable for application to large portions of the patient's body, said electrodes contribute to shielding, albeit in part, the electromagnetic field produced by the generator from the portions of the body protected by the said electrodes, and this limits the level and intensity of the induced electromagnetic currents, and consequently the effectiveness of the treatment.

In order to overcome said problem in PCT/IB2014/063413 it is described a device for treating patients which is based on a radiofrequency generator provided with one or more electrodes connected to the earth connection of an electromagnetic radiation generator—for example of the type disclosed in patent EP1301241—comprising extended electrodes formed using conductive fabrics so as to have a limited shielding effect against the electromagnetic radiation emitted by said radiofrequency generator.

Said extended electrodes can be in the form of clothing, formed using fabrics which are made conductive, so as to have a limited shielding effect against the electromagnetic radiation emitted by said radiofrequency generator.

### DETAILED DESCRIPTION OF THE INVENTION

It was now surprisingly found, and it is a first object of the present invention, that a method comprising the use of the apparatuses according to EP1301241 or PCT/IB2014/063413 (hereinafter-called Radio Electric Asymmetric Conveyer or REAC) it is possible to successfully treat neurodegenerative disorders due, for example, to Parkinson's, Alzheimer diseases and post stroke.

The REAC device generates an emission of microwaves of very weak intensity.

REAC technology is effective at very low power levels, approximately 2 mW at the emitter; however, higher powers can alter its mechanism of action. REAC technology is independent of the radiofrequency emission used. For REAC devices, the frequencies, 2.4 and 5.8 GHz, are used because they are the most widespread and are authorized at the international level (i.e. Wi-Fi). The peculiarity of REAC technology is not the emission per se, but particular physical link between the device and the patient's body and the resultant effects, which appear to be without depth limits.

The asymmetric conveyer-probe (ACP) represents this link, and the means by which this emission interacts with the biological tissues of the human body. This interaction generates radiofrequency-induced micro currents in tissues, which varies depending on the molecular characteristics of the tissue. The sum of these radiofrequency-induced micro currents produced by the instrument, and the electromagnetic field generated by the body of the subject, are concentrated by the ACP of the device and exert a therapeutic effect. Specific absorption rate (SAR) is 71 jW/kg.

According to the present invention, the method includes a set of treatment protocols.

The treatments consist in a series of combinations of pulses and pauses, each of a duration ranging from one millisecond to 100 seconds, administered for periods varying in relation to the severity of patient's status.

1-3. (canceled)

4. A method for treating a neurogenerative disease comprising administering a series of pulses and pauses to a patient in need thereof, said pauses and pulses lasting for from 1 millisecond to 100 seconds and deriving from a radio frequency generator, said radio frequency generator being equipped with at least one electrode being connected to an earth connection and said generator, and directly an electromagnetic current to said patient.

5. The method of claim 4, wherein said neurodegenerative disease is selected from the group consisting of Parkinson's disease, Alzheimer's Disease, and Post Stroke Disorder.

6. The method of claim 4, wherein said radio frequency generator is a radio electric asymmetric conveyor.
